(19) United States
(12) Patent Application Publication
(10) Pub. No.: US 2021/0057038 A1
(43) Pub. Date: Feb. 25, 2021

(54) SYSTEMS AND METHODS FOR MICROBIOME BASED SAMPLE CLASSIFICATION

(71) Applicant: PRIME DISCOVERIES, INC., New York, NY (US)

(72) Inventors: Arun Prasad MANOHARAN, New York, NY (US); Eric Shaun PROFFITT, Flushing, NY (US)

(73) Assignee: PRIME DISCOVERIES, INC., New York, NY (US)

(21) Appl. No.: 16/938,253

(22) Filed: Jul. 24, 2020

Related U.S. Application Data
(60) Provisional application No. 62/878,646, filed on Jul. 25, 2019.

(51) Int. Cl. G16B 5/20; G16B 40/00

(52) U.S. Cl. CPC G16B 5/20; G16B 40/00

(57) ABSTRACT

The classification of disease status based on the stool microbiome of the subject, or other relevant DNA, is a challenging field with a lack of accurate diagnostics. Accordingly, the inventors have developed systems and methods which accurately classify the disease status of a subject using a k-mer based algorithm for processing a subject's microbial DNA. In some examples, this includes using a logistic regression algorithm trained with L1-regularization to process DNA read derived k-mers from a subject's sample.

### CROSS-REFERENCE TO RELATED APPLICATION

This application is a 35 U.S.C. § 111(a) Utility application which claims benefit under 35 U.S.C. § 119(e) of U.S. Provisional Application No. 62/878,646 filed Jul. 25, 2019, the contents of which is incorporated herein by reference in its entirety.

### FIELD

The present invention is directed to systems and methods of classification of samples using genetic data, including to diagnose and treat subjects based on their microbiome, tissue biopsies, and other samples.

### BACKGROUND

The following description includes information that may be useful in understanding the present invention. It is not an admission that any of the information provided herein is prior art or relevant to the presently claimed invention, or that any publication specifically or implicitly referenced is prior art.

The bacteria and other microbes living in the digestive systems, skin, nasal, passages, and other locations of the body of humans and animals impact the health of their hosts in a variety of ways. For instance, these microbial communities have been shown to be related to various diseases.

Due to the physiological relationship between microbial communities and disease, many diseases are hypothesized to be associated with shifts away from a normal microbiome or are associated with certain changes in the microbiome. These include metabolic disorders, inflammatory and auto-immune diseases, neurological conditions, and cancer, among others. In particular, a number of gut-related health conditions have been studied extensively in both human and animal subjects, and there exists mounting evidence of associative and sometimes causal relationships between these conditions and the microbiome.

Current understanding of the relationship between the human microbiome and disease remains limited. Existing studies often find significant evidence for disease-associated microbial “dysbiosis”. However, there exists no comprehensive understanding of precisely how microbial communities and specific microbes within those communities cause, respond to, or contribute to disease. Accordingly, no accurate diagnostic currently exists which can determine a subject's disease using microbial biomarkers.

### SUMMARY

The methods provided herein are based, in part, on a genetic data processing method and associated algorithm that accurately classifies the disease status of a subject using a k-mer based featurization of the microbiome.

In one aspect, provided herein is a method of analyzing genetic data from a subject sample, the method comprising: receiving a subject's genetic data, wherein the genetic data comprises genetic information of bacteria present in the subject sample; and processing a sub-set of the subject's genetic data to output a set of k-mer fragments of the sub-set of the subject's genetic data.

In another aspect, provided herein is a method of analyzing genetic data from a subject sample, comprising: receiving a genetic data file comprising a set of sequences reads of bacteria present in the subject sample; sub-sampling the sequence reads to output a subset of the set of sequence reads; fragmenting the sub-set of the sequence reads using a sliding window of size K to output a set of k-mer fragments of the sub-set of the subject's genetic data and saving the subset of k-mer fragments in a table.

In another aspect, provided herein is a method of analyzing genetic data from a subject sample, the method comprising: receiving a subject's genetic data, wherein the genetic data comprises genetic information of bacteria present in the subject sample; and processing a sub-set of the subject's genetic data to output a set of k-mer fragments of the sub-set of the subject's genetic data.

In one embodiment of any of the aspects, the method further comprises processing, using a logistic regression model, at least a sub-set of the set of k-mer fragments to output an indication of whether the subject has a gastrointestinal disease.

In another embodiment of any of the aspects, the method further comprises treating the subject based on the indication of whether the subject has the gastrointestinal disease.

In another embodiment of any of the aspects, the method further comprises processing, using a logistic regression model trained with Lp regularization, the set of k-mer fragments to output an indication of whether the subject has a gastrointestinal disease.

In another embodiment of any of the aspects, the method further comprises displaying, on a display, the indication of whether the subject has a gastrointestinal disease.

In another embodiment of any of the aspects, the logistic regression model was trained with L1 regularization. In another embodiment of any of the aspects, the logistic regression model was trained with Lp regularization.

In another embodiment of any of the aspects, the at least a sub-set of k-mers was determined using stepwise regression.

In another embodiment of any of the aspects, the at least a sub-set of k-mers was determined using partial least squares regression.

In another embodiment of any of the aspects, the at least a sub-set of the set of k-mer fragments comprises each of the set of k-mer fragments.

In another embodiment of any of the aspects, the at least a sub-set of the set of k-mer fragments is determined using L1 regularization.

In another embodiment of any of the aspects, receiving the subject's genetic data further comprises: receiving a subject sample; and extracting microbial DNA from the subject sample to output the subject's genetic data.

In another embodiment of any of the aspects, the subject sample comprises at least one of the following: a swab sample, a swab stool sample, a swab buccal sample, a swab nasal sample, vaginal swab, a swab saliva sample, a urine sample, or a blood sample.

In another embodiment of any of the aspects, the gastrointestinal disease comprises at least one of the following: Crohn's Disease, Ulcerative Colitis, C. difficile infection, Severe Ulcerative Colitis, Moderate Ulcerative Colitis, inactive Ulcerative Colitis, or Anorexia.

In another embodiment of any of the aspects, processing the subset of the subject's genetic data to output a set of k-mer fragments of the subset of the subject's genetic data further comprises determining a frequency of occurrence of each of the set of k-mer fragments.

In another embodiment of any of the aspects, the set of k-mer fragments comprise 2-mers, 3-mers, 4-mers, 5-mers, 6-mers, 7-mers, 8-mers, 9-mers, 10-mers, 11-mers, 12-mers.

In another embodiment of any of the aspects, the genetic information of bacteria comprises DNA.

In another embodiment of any of the aspects, the step of receiving the subject's genetic data comprises receiving a FASTQ file with sequence reads from a sample from the subject.

In another embodiment of any of the aspects, processing a sub-set of the subject's genetic data to output a set of k-mer fragments comprises using a sliding window on the sequence reads from the FASTQ file.

In another embodiment of any of the aspects, the step of processing a sub-set of the subject's genetic data to output a set of k-mer fragments further comprises outputting a normalized vector representing the relative frequency of occurrence of each k-mer.

In another embodiment of any of the aspects, the subject comprises a human or animal.

In another embodiment, the sub-sampling is performed randomly.

In another embodiment, Lp regularization comprises elastic net regularization.

In another embodiment, Lp regularization comprises L1 regularization, L1.001, regularization, or L1.002 regularization.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 depicts an example of an overview of a system according to some embodiments of the present disclosure;

FIG. 2 depicts a flow chart showing an example process for implementing a diagnostic according to the present disclosure;

FIG. 3 depicts a diagram showing an example process for extracting k-mers;

FIG. 4 depicts a diagram showing an example process for normalizing k-mers;

FIG. 5 depicts a graph showing experimental results from an example of the disclosed classifier to distinguish IBS samples from controls;

FIG. 6 depicts a graph showing experimental results from an example of the disclosed classifier to distinguish between Crohn's disease, ulcerative colitis, and controls;

FIG. 7 depicts a graph showing experimental results from an example of the disclosed classifier to distinguish Crohn's disease from ulcerative colitis;

FIG. 8 depicts a graph showing experimental results from an example of the disclosed classifier to distinguish Crohn's disease from control subjects;

FIG. 9 depicts a graph showing experimental results from an example of the disclosed classifier to distinguish C. difficile infected from control subjects;

FIG. 10 depicts a graph showing experimental results from an example of the disclosed classifier to distinguish moderate/severe ulcerative colitis from inactive ulcerative colitis;

FIG. 11 depicts a graph showing experimental results from an example of the disclosed classifier to distinguish colorectal cancer from control tissue;

FIG. 12 depicts a graph showing experimental results from an example of the disclosed classifier to distinguish anorexic from control subjects;

FIG. 13 depicts a graph showing experimental results from an example of the disclosed classifier to distinguish sample type;

FIG. 14 depicts a graph showing experimental results from an example of the disclosed classifier to distinguish body sample site; and

FIG. 15 depicts a graph showing experimental results from an example of the disclosed classifier to distinguish animal sample source.

In the drawings, the same reference numbers and any acronyms identify elements or acts with the same or similar structure or functionality for ease of understanding and convenience. To easily identify the discussion of any particular element or act, the most significant digit or digits in a reference number refer to the figure number in which that element is first introduced.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Definitions

As used herein, a “gastrointestinal disease” means any gut-related disease, disorder, or health condition.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” may be used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a gastrointestinal disease or inflammatory bowel disease (IBD). A subject can be male or female. A subject can be of any age. For example, a subject can be an adult, a child, an infant, or a neonate.

A subject can be one who has been previously diagnosed with or identified as suffering from or having a condition in need of treatment (e.g. a gastrointestinal disease or disorder, IBD, Crohn's disease, ulcerative colitis) or one or more complications related to such a condition, and optionally, have already undergone treatment for such disease or the one or more complications related thereto. Alternatively, a subject can also be one who has not been previously diagnosed as having a gastrointestinal disease or disorder (e.g., IBD, Crohn's disease, ulcerative colitis) or one or more complications related to such a condition. For example, a subject can be one who exhibits one or more risk factors for a gastrointestinal disease or disorder (e.g., IBD, Crohn's disease, ulcerative colitis) or one or more complications related thereto or a subject who does not exhibit risk factors.

As described herein, a subject can be a pediatric subject. The human gut microbiome changes dramatically from birth to adulthood, but the human gut microbiome matures faster than the rest of the individual; that is, in many ways, after age 3 years, the healthy human gut microbiome tends to be very similar to that of an adult human. Thus, as used herein, the term “pediatric,” when used in reference to the gut microbiome or to gastrointestinal disease status, refers to a human subject or patients from birth to three years of age.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g., a gastrointestinal disease or disorder, e.g. IBD, Crohn's disease, or ulcerative colitis. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments as described herein, nucleic acid sequence data can be obtained via high-throughput sequencing in the format provided by different sequencing platforms that output raw genetic data. As a non-limiting example, nucleic acid sequence data can be provided in at least one of the following formats: raw sequence read format, plain sequence format, Federal Acquisition Streamlining Act-All (FASTA) format, FASTA Quality score (FASTQ) format, European Molecular Biology Laboratory (EMBL) format, binary base call (BCL) format, Variant Call Format (VCF), Binary Alignment Map (BAM) format, Sequence Alignment Map (SAM) format, Wisconsin GCG format, GCG-Rich Sequence Format (GCG-RSF), GenBank format, IG format, CRAM format, Standard Flowgram Format (SFF), Hierarchical Data Format (HDF; e.g., HDF4, HDF5), Color Space FASTA (CSFASTA) format, Sequence Read Format (SRF), Native Illumina format, or QSEQ format.

### Overview

Classification of a subject's disease status based on a subject's microbiome, biopsy samples, or other relevant DNA sample is a challenging field with a lack of accurate diagnostics available. Accordingly, the inventors have developed a genetic data processing method and associated algorithm which accurately classifies the disease status of a subject using a k-mer based featurization of the microbiome. In some examples, this includes using a logistic regression algorithm trained with L1-regularization (also known as least absolute shrinkage and selection operator “LASSO”) to process the k-mers. In some examples, k-mers are processed from a subset of the sequencing reads from the microbiome sample.

The samples may be from a variety of sources, and may include skin swabs, buccal swabs, fecal swabs, vaginal swabs, nasal swabs, biopsies, saliva, urine, or blood. The classifier may be used as a diagnostic for a variety of diseases including:
Crohn's Disease
Ulcerative Colitis
C. difficile infection;
Anorexia;
IBD generally;
Irritable Bowel Syndrome (IBS);
Severe/Moderate Ulcerative Colitis;
Inactive Ulcerative Colitis;
Colorectal Cancer; and
Others.

The classifier performs well across a variety of indications and sample sources, and thus is an unexpectedly robust classifier given the data and as described below. For instance, other researchers have tried and received subpar results using other types of classifier algorithms including: (1) Support Vector Machines, (2) Random Forest, and (3) Deep Learning (e.g., multi-layer perceptrons). See, e.g., Asgari et al, 2018, “MicroPheno: Predicting environments and host phenotypes from 16S rRNA gene sequencing using a k-mer based representation of shallow sub-samples.” Accordingly, the type of algorithm and pre-processing methods (e.g. k-mers) are very important for the accuracy in some examples.

### System

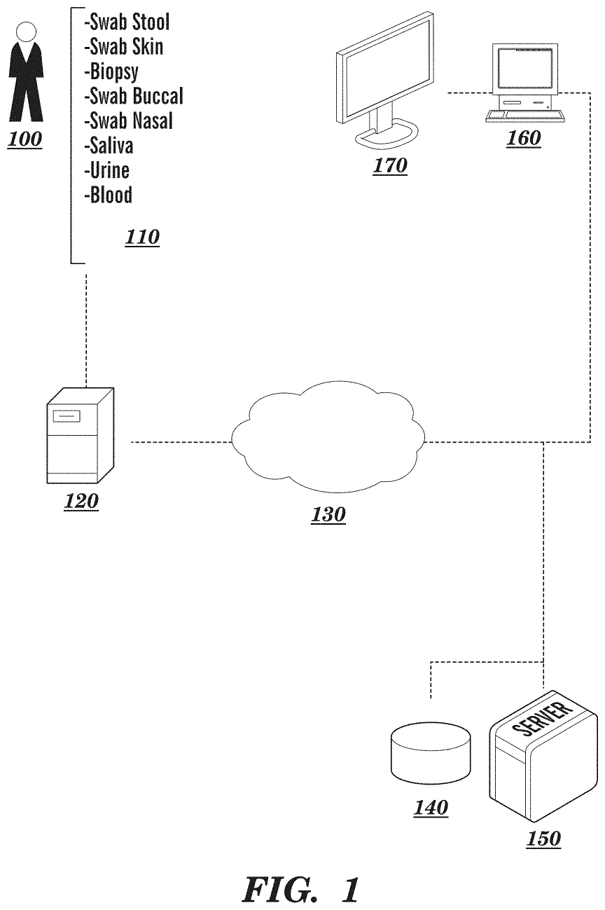

FIG. 1 illustrates an example overview of a system for implementing the current disclosure. The system may include a subject 100 and a variety of subject samples 110 that may include:
stool swabs;
skin swabs;
buccal swabs;
nasal swabs;
biopsies;
saliva;
urine;
blood samples; and
other suitable samples from the subject that may contain bacteria.

Additionally, the system includes a gene sequencer 120 for processing the genetic information in samples from the subject. The gene sequencer 120 may be any suitable sequencer for determining the DNA sequences of the bacteria contained in the samples 110 from the subject 100 or the DNA of the biopsied or collected tissue. For instance, suitable gene sequencing systems may include the MiSeq, NextSeq, HiSeq, NovaSeq, Oxford Nanopore, and PacBio sequencers. However, additional sequencing technologies that are suitable may be utilized, for instance as disclosed by Osman in a 2018 paper titled “16S rRNA Gene Sequencing for Deciphering the Colorectal Cancer Gut Microbiome: Current Protocols and Workflows,” the contents of which is incorporated by reference herein in its entirety, including but not limited to the examples it discloses for other steps and systems utilized for sequencing herein.

The gene sequencer 120 may be connected to a network 130. Network 130 may be an internal network, external network, the internet or any other system or method for electronic communication. In other examples, the data may be manually removed from gene sequencer 120.

Network 130 may be connected to computing device 160 and display 170. Computing device 160 may be any suitable computing device 160, including a desktop computer, server (including remote servers), mobile device, or other suitable computing device 160. Additionally, network 130 may be connected to a server 150 and database 140. In some examples, algorithms, and other software may be stored in database 140 and run on server 150. Additionally, subject 100 data and other genetic information may be stored in database 140.

### Methods—Sequencing Samples

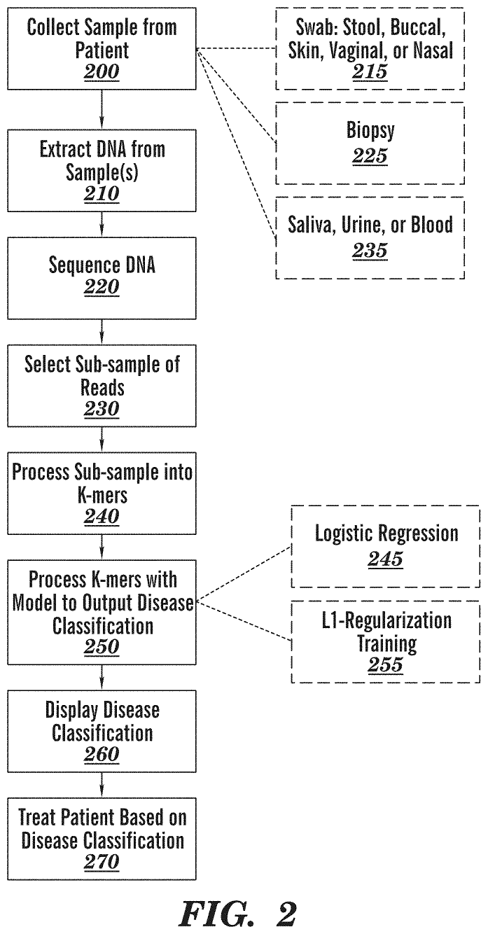

FIG. 2 illustrates an example of a method for classifying a subject's 100 sample 110 and treating a subject 100. For instance, first a sample 110 may be collected from a subject 200. This may be performed by a caregiver using any suitable methods, including swabs 215, biopsies 225, or collection of saliva, urine, blood, tears, or other bodily fluids 235.

Next, the DNA from the sample 110 may be extracted 210 using any suitable techniques that would allow sequencing of the DNA. For instance, a variety of protocols could be utilized that involve cellular lysis, non-DNA macromolecule elimination together with DNA detachment and collection as disclosed in Osman in a 2018 paper titled “16S rRNA Gene Sequencing for Deciphering the Colorectal Cancer Gut Microbiome: Current Protocols and Workflows,” the contents of which is incorporated by reference herein in its entirety. In some examples, a quality control step may be performed and the DNA may be resampled or reextracted if quality control fails. Additionally, the DNA may be prepared for sequencing for a variety of methods, including 16S ribosomal RNA sequencing, shallow shotgun sequencing, WGS shotgun sequencing or other suitable sequencing methods.

Next, the prepared DNA may be sequenced 220 with a variety of methods to output a data file containing all of the sequence reads. For instance, the prepared DNA may be processed with a high throughput sequencer, to output a FASTQ/FASTA file or other file containing raw genetic information. Examples of this are provided by Osman, in a 2018 paper titled “16S rRNA Gene Sequencing for Deciphering the Colorectal Cancer Gut Microbiome: Current Protocols and Workflows,” the contents of which is incorporated herein in its entirety.

Then, the sequence data may be transmitted over a network 130 to be stored in a database 140 by a server 150. In some examples, the server 150 may then perform further processing on the sequence data or sequence data files.

Methods—Processing Sequence Data into k-Mers

For instance, a variety of steps may be performed to select a sub-sample of the reads 230 that includes, QC, random sampling and other processes. For instance, sequences may be de-multiplexed into samples, and samples that fail QC may be removed. In some examples, sequences may be de-noised, chimeric sequences may be removed, and sequences outside targeted regions (if known) may be removed. In some examples, a sub-sampling (depth) value will be set to a positive integer denoting the number of reads to be sub-sampled from the FASTQ/FASTA file. Accordingly, after the sub-sampling process is applied, the output would be set of sub-sampled reads equal in number to the specified depth.

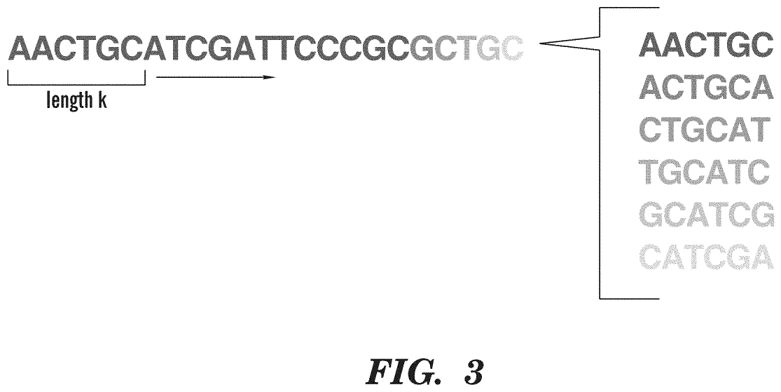

Next, the server 150 or other processor may process the sub-sampled reads into k-mers 240. For instance, the server 150 may process the reads in the FASTQ or other sequence file using a sliding window of length “k” as illustrated in FIG. 3. In some examples, this process does not concatenate the reads, but rather, starts the sliding window fresh at the beginning of each new read.

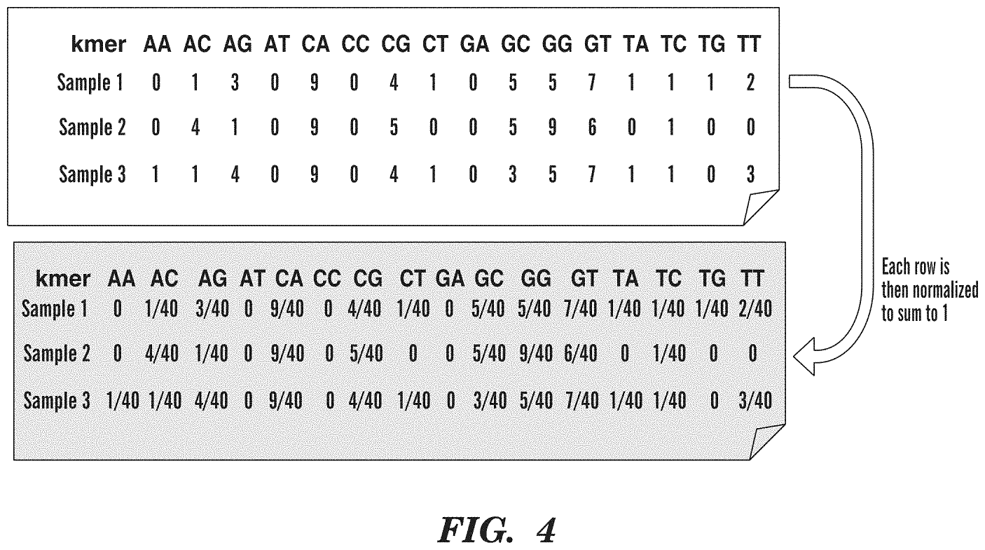

In some examples, the number of k-mer fragments corresponding to each of the 4^{k} unique k-mers is counted for each sample 110, and these counts are assembled into a vector. For example, FIG. 4 illustrates an example process for determining the frequency of each k-mer in each sub-sample and outputting a vector containing the processed reads of the sample 110 from the subject 100. In some examples, the k-mer vector may be normalized so that it sums to 1 by dividing by each component by the sum of the counts across all k-mers.

In some examples, the sliding window length “k” could be 2, 3, 4, 5, 6, 7, 8, 9, 10. 11. 12 or other suitable numbers.

Methods—Inputting k-Mer Data into Model to Output Classification

Next, the vectors or other processed k-mer data may input into a trained model to output a disease classification 250 of the subject 100 based on the DNA from the sample. In some examples, this model will be a logistic regression model 245. The logistic regression model may be trained with L1-regularization 255 or other training methods that identify or promote a sub-set of k-mers for processing. For instance, the logistic regression model may be trained with Lₚ regularization where “p” is defined as a real number greater than or equal to “1” that when applied effectively identifies or promotes a sub-set of k-mers using the same technique as L1 regularization but replaces “1” with a real number greater than “1.” Accordingly, by way of example only, Lₚ regularization includes but is not limited to: L1, L1.001, L1.002, and L2 regularization. In some examples, Lₚ regularization may include linear combinations of different regularizations (e.g. ½ L1+½ L2 regularization). In other examples, different techniques may be utilized to weight or promote the most relevant sub-set of k-mers (e.g. feature selection).

Next, the disease classification 260 may be displayed on the display 170. This could be in the form of a particular disease name, the probability that a subject has a disease, a disease the subject does not have or other suitable indication of the disease. In some examples, the display 170 may indicate a suitable treatment or treatment course for the disease.

Additionally, a caregiver may treat the subject 100 based on the disease classification 270. For instance, Table 1 indicates classification outputs and potential, exemplary treatments that could be administered to the subject by the caregiver. However, these treatments are only examples, and one of skill in the art would understand that additional suitable treatments may be available for these diseases.

TABLE 1
Disease Classifications Output from disclosed
Classifiers and Examples of Potential Treatments
for the Disease and/or its Symptoms

| Disease | |
|---|---|
| Classification Output | Example Treatments |
| Irritable Bowel | Alosetron, Eleuxadoline, |
| Disease | Rifaximin, Lubiprostone, |
| | Linaclotide, fiber, laxatives, pain |
| | medications, antidepressants, |
| | anticholinergic medications, |
| | anti-diarrheal medications, and |
| | diet changes. |
| Ulcerative Colitis | Anti-inflammatory drugs |
| | (e.g. 5-amiosalicylates), immune |
| | system suppressors |
| | (e.g. Azathioprine, Cyclosporine, |
| | Infliximab, Vedolizumab), antibiotics, |
| | anti-diarrheal, pain relievers, |
| | iron supplements, and surgery. |
| C. difficile | Antibiotics, fecal microbiota |
| | transplant, probiotics, and surgery. |
| Colorectal Cancer | Surgery, chemotherapy, radiation, |
| | therapy, immunotherapy and, |
| | proton beam therapy. |
| Anorexia | Therapy, diet changes, antidepressants, or |
| | other psychiatric medications. |

### EXAMPLES

The following examples are provided to better illustrate the claimed invention and are not intended to be interpreted as limiting the scope of the invention. To the extent that specific materials or steps are mentioned, it is merely for purposes of illustration and is not intended to limit the invention. One skilled in the art may develop equivalent means or reactants without the exercise of inventive capacity and without departing from the scope of the invention.

Generally, the efficacy of the classifiers disclosed herein has been established on a variety of public datasets and has returned accurate classification results across a variety of sample types and gut-related health conditions. Certain studies associated with these public datasets have either developed their own or leveraged existing classifiers to make the same type of predictions as disclosed herein. However, in those instances (described below), the disclosed classifier showed far superior performance to the extent that the results could be compared given the information that was made publicly available in these studies.

Accordingly, the classifier performed well across multiple disease phenotypes, underscoring its suitability as a panel diagnostic. In addition, the classifier's robustness to sample type suggests its utility in verifying/identifying the DNA source and host.

### Example 1: IBS Versus Control

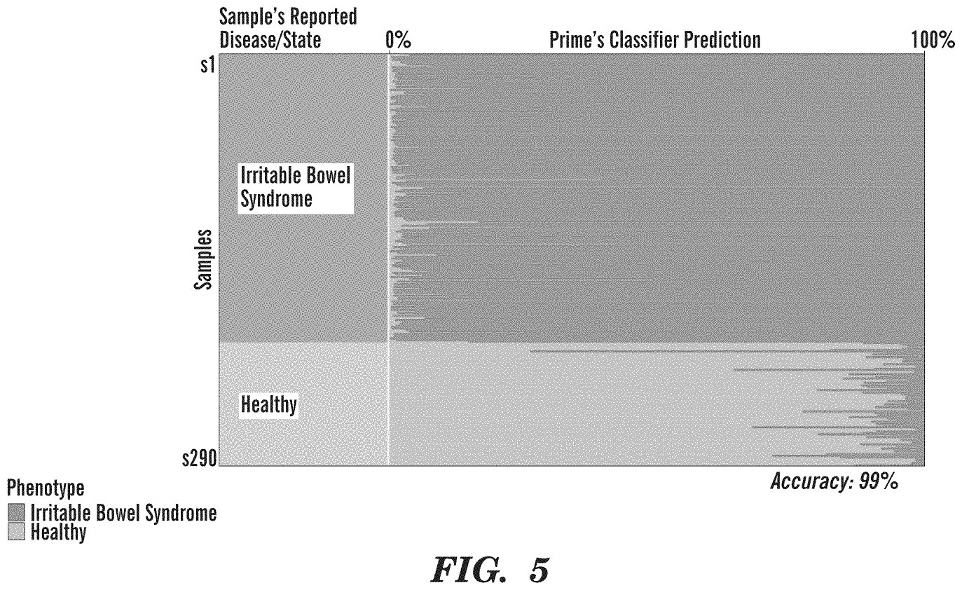

FIG. 5 illustrates an example of the disclosed classifier which used the fecal microbiome to distinguish IBS samples from control with an accuracy of 99%. The sequencing and metadata retrieved from publicly available data was published as part of the 2015 study by Pozuelo et al., “Reduction of butyrate- and methane-producing microorganisms in subjects with Irritable Bowel Syndrome,” which is incorporated by reference herein in its entirety. The study did not disclose attempts to classify subjects using a classifier based on their DNA.

### Example 2: Crohn's Disease Vs. Ulcerative Colitis Vs. Control

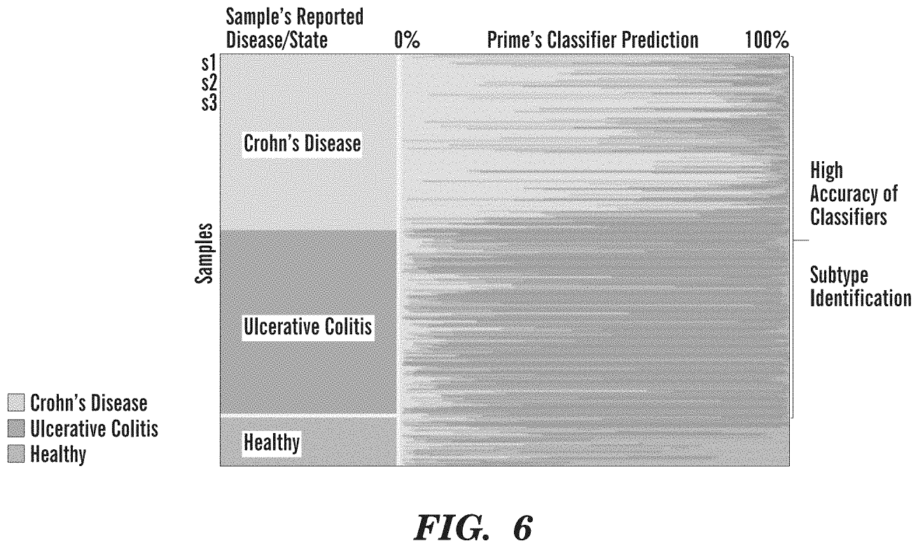

FIG. 6 illustrates an example of the disclosed classifier applied to classify subjects into groups of controls, ulcerative colitis, and Crohn's disease with an accuracy of 83.8% from fecal sample DNA. Additionally, the disclosed classifier was applied to classify subjects into groups of controls and IBD (by grouping the ulcerative colitis and Crohn's disease samples together) with an accuracy of 94.2% The DNA and subject disease labels were retrieved from publicly available data from the 2017 study by Halfvarson et al., “Dynamics of the human Gut Microbiome in Inflammatory Bowel Disease,” the contents of which are incorporated herein by reference in its entirety. In that paper, the authors used a random forest model to classify subjects into groups of IBD subtypes and controls, but only achieved an accuracy of 66.6%. Accordingly, it appears the disclosed classifier achieved far superior results with the same data set and illustrates the importance of the classification model and processing steps in achieving high accuracy.

### Example 3: Crohn's Vs. Ulcerative Colitis

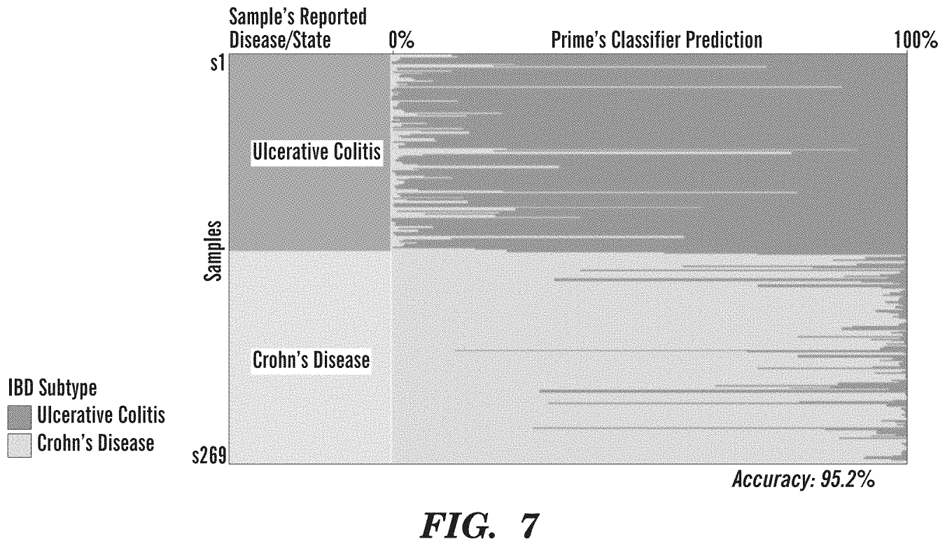

FIG. 7 illustrates an example of the disclosed classifier applied to classify subjects into groups of ulcerative colitis and Crohn's disease from fecal sample DNA. The classifier had an accuracy of 95.2%. The DNA and subject disease known labels were retrieved from publicly available data from the 2017 study by Pascal et al., “A microbial signature for Crohn's disease,” the contents of which are incorporated herein by reference in its entirety. The study did not disclose attempts to classify subjects into groups of ulcerative colitis and Crohn's disease.

### Example 4: Crohn's Disease Vs. Controls

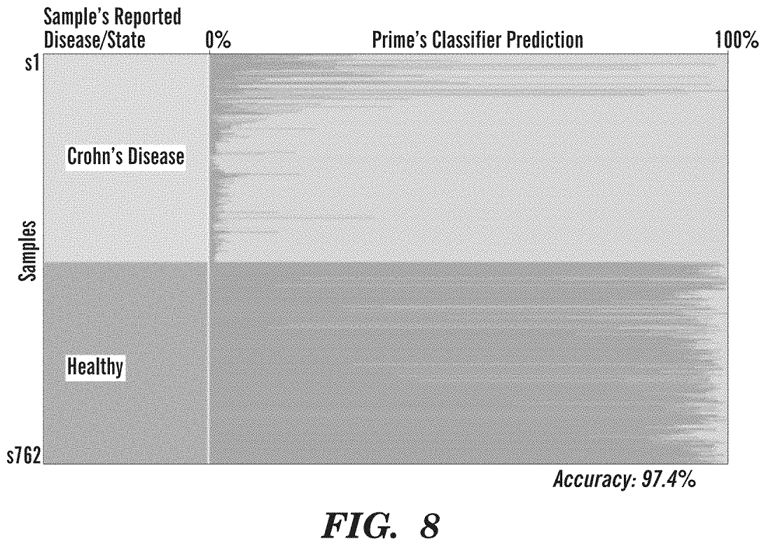

FIG. 8 illustrates an example of the disclosed classifier applied to classify subjects into groups of Crohn's disease and control from fecal sample DNA. The classifier had an accuracy of 97.4% and an AUC of 0.988. The DNA and subject disease known labels were retrieved from publicly available data from the 2017 study by Vazquez-Baeza et al., “Guiding longitudinal sampling in IBD cohorts,” the contents of which are incorporated herein by reference in its entirety. The study disclosed a comparable classifier which only achieved an AUC of 0.8.

### Example 5: C. difficile Vs. Controls

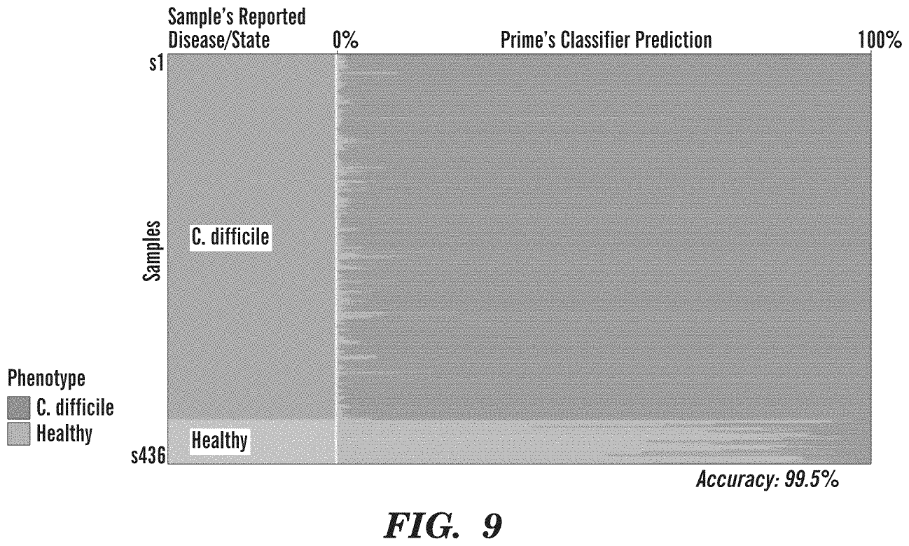

FIG. 9 illustrates an example of the disclosed classifier applied to classify subjects into groups infected with C. difficile and controls from fecal sample DNA. The classifier had an accuracy of 99.5%. The DNA and subject disease known labels were retrieved from publicly available data from the 2018 study by Thorpe et al., “Enhanced preservation of the human intestinal microbiota by ridinilazole, a novel Clostridium difficile-targeting antibacterial, compared to vancomycin,” the contents of which are incorporated herein by reference in its entirety. The study did not disclose attempts to classify subjects using a classifier based on their DNA.

### Example 6: Severity of Ulcerative Colitis

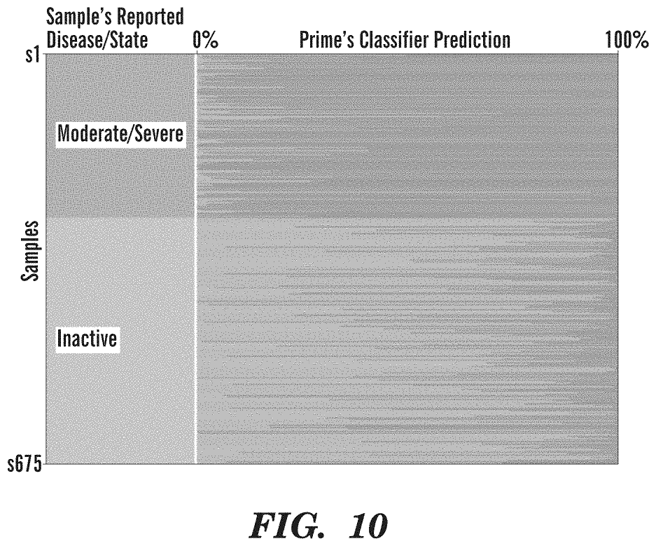

FIG. 10 illustrates an example of the disclosed classifier applied to classify pediatric subjects into groups with different stages or severities of ulcerative colitis—moderate severe (PUCAI>34) and inactive (PUCAI<10) from fecal sample DNA. The classifier had an accuracy of 86%. The DNA and subject disease known labels were retrieved from publicly available data from the 2018 study by Xavier et al., “Compositional and Temporal Changes in the Gut Microbiome of Pediatric Ulcerative Colitis Patients are Linked to Disease Course,” the contents of which are incorporated herein by reference in its entirety. The study did not disclose attempts to classify subjects using a classifier based on their DNA.

### Example 7: Colonic Tumor Tissue Vs. Adjacent Normal Tissue

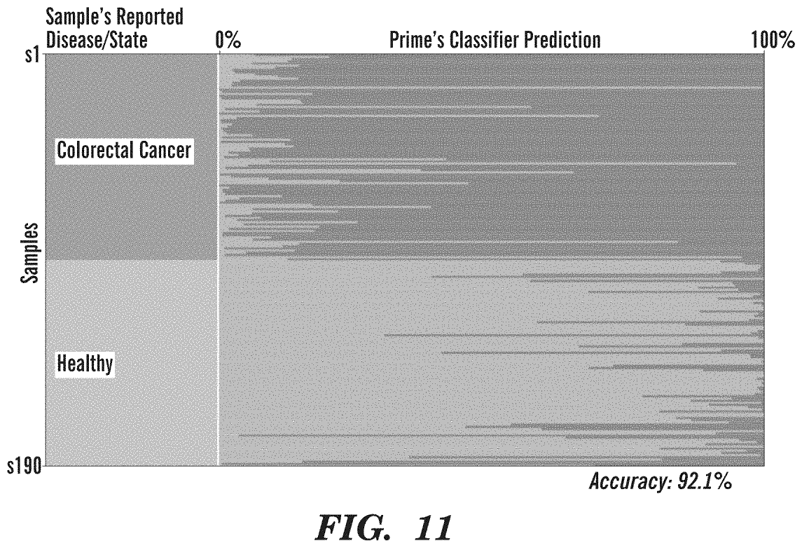

FIG. 11 illustrates an example of the disclosed classifier applied to classify biopsied tissue into groups of colonic tumor tissue and adjacent normal tissue from tissue sample biopsy bacterial DNA. The classifier had an accuracy of 92.1%. The DNA and subject disease known labels were retrieved from publicly available data from the 2012 study by Xavier et al., “Genomic Analysis identifies association of Fusobacterium with Colorectal Carcinoma,” the contents of which are incorporated herein by reference in its entirety. The study did not disclose attempts to classify subjects using a classifier based on their DNA.

### Example 8: Anorexia Vs. Controls

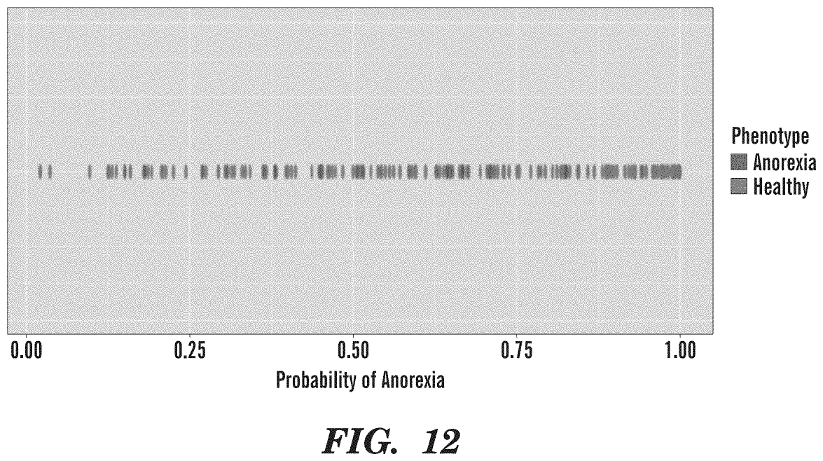

FIG. 12 illustrates an example of the disclosed classifier applied to classify samples from subjects that had anorexia vs. control subjects from fecal sample DNA. The classifier had an accuracy of 78.5%. The DNA and subject disease known labels were retrieved from publicly available data from the 2016 study by Mack et al., “Weight gain in Anorexia Nervosa does not Ameliorate the Fecal Microbiota Branched Chain Fatty Acid Profiles, and Gastrointestinal Complaints,” the contents of which are incorporated herein by reference in its entirety. The study did not disclose attempts to classify subjects using a classifier based on their DNA.

### Example 9: Sample Sources

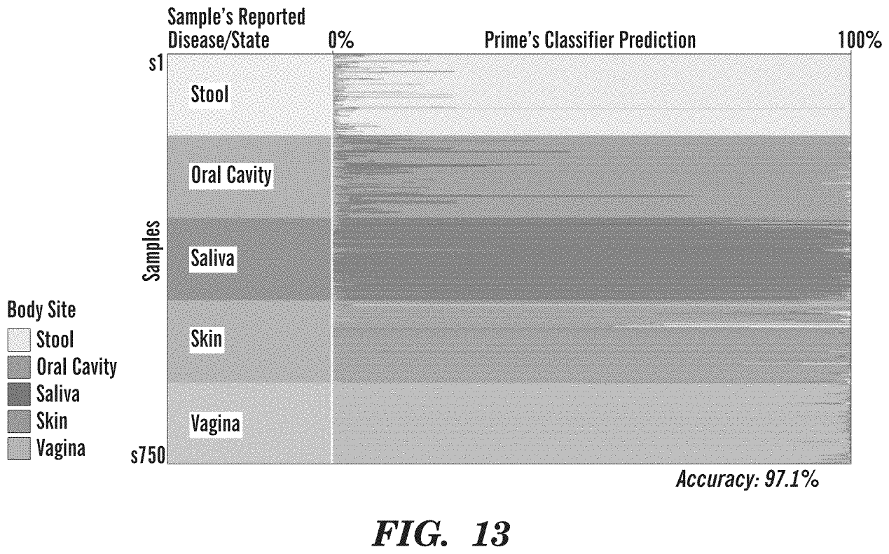

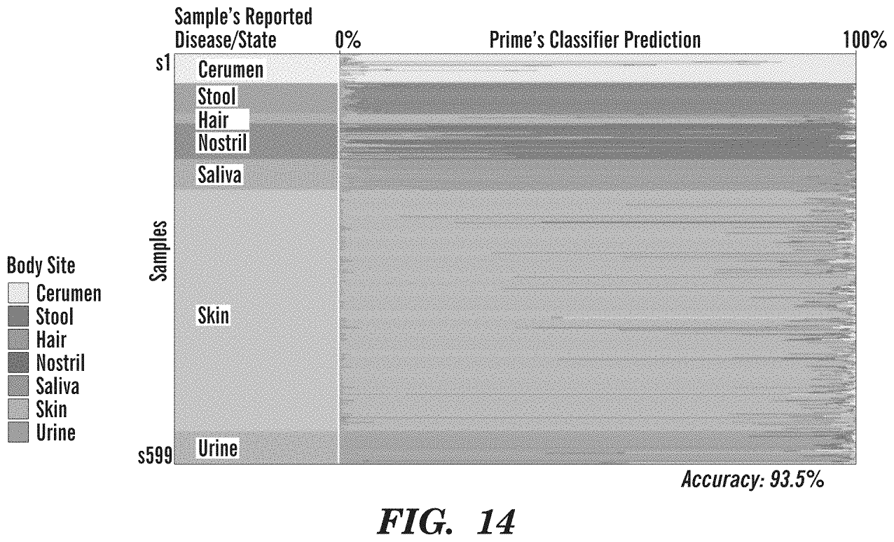

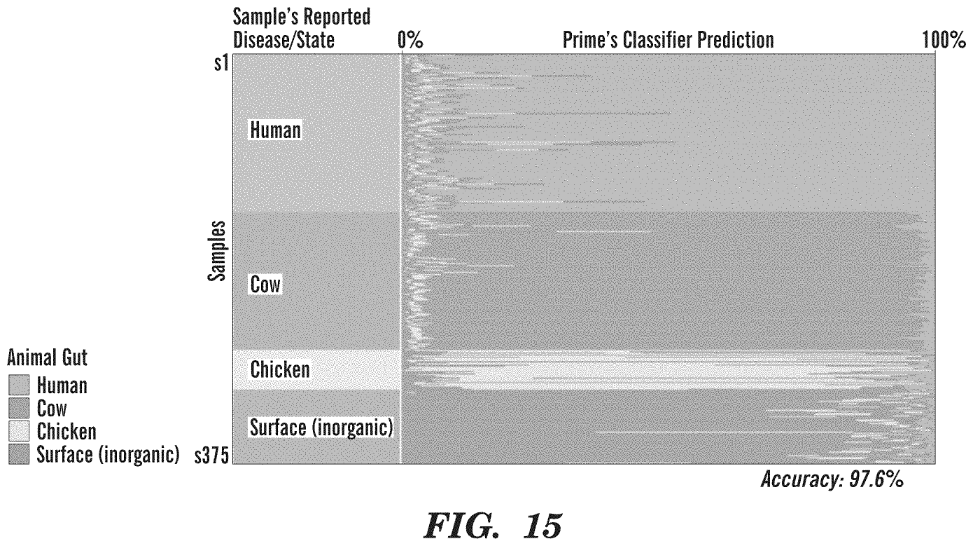

FIGS. 13-15 illustrate examples of the disclosed classifier applied to classify the source of the samples. All had accuracies in the range of 93-98%. The DNA and sample source known labels were retrieved from various publicly available data. FIG. 13 illustrates the results of one example of the classifier applied to distinguish body sites of the sample. FIG. 14 illustrates the results of one example of the classifier applied to distinguish sample types. FIG. 15 illustrates the results of one example of the classifier applied to distinguish animal sources of the sample.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively, or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a Read-Only Memory or a Random Access Memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### CONCLUSION

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A method of analyzing genetic data from a subject sample, the method comprising:
receiving a subject's genetic data, wherein the genetic data comprises genetic information of bacteria present in the subject sample;
processing a sub-set of the subject's genetic data to output a set of k-mer fragments of the sub-set of the subject's genetic data; and
processing, using a logistic regression model, at least a sub-set of the set of k-mer fragments to output an indication of whether the subject has a gastrointestinal disease; and
treating the subject based on the indication of whether the subject has the gastrointestinal disease.

2. The method of claim 1, wherein the logistic regression model was trained with L1 regularization.

3. The method of claim 1, wherein the at least a sub-set of k-mers was determined using stepwise regression.

4. The method of claim 1, wherein the at least a sub-set of k-mers was determined using partial least squares regression.

5. The method of claim 1, wherein the logistic regression model was trained with Lₚ regularization.

6. The method of claim 1, wherein the at least a sub-set of the set of k-mer fragments comprises each of the set of k-mer fragments.

7. The method of claim 1, wherein the at least a sub-set of the set of k-mer fragments is determined using L1 regularization.

8. The method of claim 1, wherein receiving the subject's genetic data further comprises:
receiving a subject sample; and
extracting microbial DNA from the subject sample to output the subject's genetic data.

9. The method of claim 1, wherein the subject sample comprises at least one of the following: a swab sample, a swab stool sample, a swab buccal sample, a swab nasal sample, vaginal swab, a swab saliva sample, a urine sample, or a blood sample.

10. The method of claim 1, wherein the gastrointestinal disease comprises at least one of the following: Crohn's Disease, Ulcerative Colitis, C. difficile infection, Severe Ulcerative Colitis, Moderate Ulcerative Colitis, inactive Ulcerative Colitis, or Anorexia.

11. The method of claim 1, wherein processing the subset of the subject's genetic data to output a set of k-mer fragments of the subset of the subject's genetic data further comprises determining a frequency of occurrence of each of the set of k-mer fragments.

12. The method of claim 1, wherein the set of k-mer fragments comprise 2-mers, 3-mers, 4-mers, 5-mers, 6-mers, 7-mers, 8-mers, 9-mers, 10-mers, 11-mers, 12-mers.

13. The method of claim 1, wherein the genetic information of bacteria comprises DNA.

14. The method of claim 1, wherein receiving the subject's genetic data comprises receiving a FASTQ file with sequence reads from a sample from the subject.

15. The method of claim 14, wherein processing a sub-set of the subject's genetic data to output a set of k-mer fragments comprises using a sliding window on the sequence reads from the FASTQ file.

16. The method of claim 1, wherein processing a sub-set of the subject's genetic data to output a set of k-mer fragments further comprises outputting a normalized vector representing the relative frequency of occurrence of each k-mer.

17. The method of claim 1, wherein the subject comprises a human or animal.

18. A method of analyzing genetic data from a subject sample, comprising:
receiving a genetic data file comprising a set of sequences reads of bacteria present in the subject sample;
sub-sampling the sequence reads to output a subset of the set of sequence reads;
fragmenting the sub-set of the sequence reads using a sliding window of size K to output a set of k-mer fragments of the sub-set of the subject's genetic data and saving the subset of k-mer fragments in a table; and
processing, using a logistic regression model trained with Lₚ regularization, the set of k-mer fragments to output an indication of whether the subject has a gastrointestinal disease; and
displaying, on a display, the indication of whether the subject has a gastrointestinal disease.

19. The method of claim 18, further comprising treating the subject if the patient has a gastrointestinal disease.

20. The method of claim 18, wherein the sub-sampling is performed randomly.

21. The method of claim 18, wherein Lₚ regularization comprises elastic net regularization.

22. The method of claim 18, wherein Lₚ regularization comprises L1 regularization, L1.001, regularization, or L1.002 regularization.

23. A method of analyzing genetic data from a subject sample, the method comprising:
receiving a subject's genetic data, wherein the genetic data comprises genetic information of bacteria present in the subject sample;
processing a sub-set of the subject's genetic data to output a set of k-mer fragments of the sub-set of the subject's genetic data; and
processing, using a logistic regression model, at least a sub-set of the set of k-mer fragments to output an indication of whether the subject has a gastrointestinal disease; and
displaying, on a display, the indication of whether the subject has a gastrointestinal disease.

24. The method of claim 23, wherein the logistic regression model was trained with L1 regularization.

25. The method of claim 23, wherein the at least a sub-set of k-mers was determined using stepwise regression.

26. The method of claim 23, wherein the at least a sub-set of k-mers was determined using partial least squares regression.

27. The method of claim 23, wherein the logistic regression model was trained with Lₚ regularization.

28. The method of claim 23, wherein the at least a sub-set of the set of k-mer fragments comprises each of the set of k-mer fragments.

29. The method of claim 23, wherein the at least a sub-set of the set of k-mer fragments is determined using L1 regularization.


SEQUENCE LISTING (1 sequence)

SEQ ID NO: 1 - DNA, 23 bp, Artificial Sequence
  Description of Artificial Sequence Synthetic oligonucleotide
aactgcatcgattcccgcgctgc